(19) United States
(12) Patent Application Publication
(10) Pub. No.: US 2024/0292867 A1
(43) Pub. Date: Sep. 5, 2024

(54) A METHOD FOR THE PRODUCTION OF PRUNE JUICE CONCENTRATE

(71) Applicant: TUNAY GIDA SANAYI VE TICARET ANONIM SIRKETI, ERZINCAN (TR)

(72) Inventor: Gonca KIRTILOGLU DEMIR, ERZINCAN (TR)

(21) Appl. No.: 18/575,800

(22) PCT Filed: Apr. 15, 2022
(86) PCT No.: PCT/TR2022/050342

§ 371 Date: Dec. 29, 2023

(30) Foreign Application Priority Data
Jun. 29, 2021 (TR) TR2021/010587

(51) Int. Cl. A23L 2/08; A23L 2/46; A23L 2/74; A23L 2/84

(52) U.S. Cl. CPC A23L 2/087; A23L 2/46; A23L 2/74; A23L 2/84

(57) ABSTRACT

Disclosed herein is a method for producing prune juice concentrate. By the such method of the invention, a production of prune juice concentrate with low acidity and low cost is achieved by using sweet plum varieties such as President, Stanley, Sugar, and Uryani with high purity.

This application is a National Phase entry of International Application No. PCT/TR2022/050342 under § 371 and claims the benefit of Turkish Patent Application No. TR2021/010587, filed Jun. 29, 2021, which is hereby incorporated by reference in its entirety.

### FIELD OF THE DISCLOSURE

The present disclosure relates to a method for producing prune juice concentrate. With the method of at least one embodiment of the invention, a production of prune juice concentrate with high purity and low cost is achieved.

### BACKGROUND

Plum is in the Rosaceae family. With peels being thin and the size of a walnut, plum is a fruit rich in vitamin B. It is cultivated as well as grown wild. Plum, which is a source of vitamins A and C, copper minerals, potassium, and fiber, also contains phosphorus and iron.

Prunes are obtained by drying the plum in the sun or by industrial processes. Prunes protect the health of the body in general, as well as eliminate some health problems that arise. It is beneficial for the digestive system due to its fiber content. Vitamin K, which it contains in large amounts, prevents blood clotting and protects the nervous system. Vitamin A in its content is beneficial for eye health. Prunes are also antioxidants, and with this feature, they protect the immune system and reduce the risk of getting sick.

It is quite common to obtain prune juice in order to make prunes easier to use by consumers by preserving all these benefits of them. While prune juice is beneficial for the digestive system, bone health, and heart health, it is a great source of iron and helps prevent and treat iron deficiency.

Due to all these benefits, prune juice production attracts a lot of attention today and various studies are being carried out to develop processes that are low in cost and easy to implement in the state of the art.

In the patent application numbered CN105661198A, a process for preparing a prune juice concentrate is presented. The process steps of the invention include raw material processing, enzymatic hydrolysis, homogenization and sterilization steps.

As a result, all the above-mentioned problems have made it necessary to make an innovation in the relevant technical field.

### SUMMARY

It is an object of embodiments of the present invention to provide a method for obtaining a cost-effective product by using sweet plum varieties such as President, Stanley, Sugar, Uryani, with high purity, as an alternative to the prune juice concentrate preparation processes in the art.

In order to achieve all the above-mentioned objectives and objectives which will emerge from the following detailed description, at least one embodiment of the present invention relates to a method for producing prune juice concentrate. Accordingly, such method includes the following steps:
a. supplying prunes,
b. subjecting the prunes to the cleaning process,
c. softening prunes by adding water,
d. conveying the products obtained in step c to the mash tank,
e. obtaining raw fruit juice by pressing the products conveyed to the press from the mash tank,
f. applying coarse filtration to the juice obtained in step e,
g. carrying out the pasteurization process,
h. carrying out the enzymatic process,
i. carrying out the clarification process,
j. carrying out the rotary vacuum filtration,
k. carrying out the ultra filtration,
l. taking the filtered product into clear tanks,
m. carrying out the evaporation,
n. cooling the concentrated product obtained in step m,
o. mixing the cooled product,
p. performing the filling.

In one embodiment of the invention, the amount of water in step c of the method is twice the amount of the mentioned prune by weight.

In one embodiment of the invention, the softening process in step c of the method is carried out by heating the mentioned prunes at 50-80° C., preferably 60-70° C. for 100-140 minutes and preferably 120 minutes.

In one embodiment of the invention, the products conveyed to the mash tank in step d of the method are kept here at 40-70° C., preferably 50-60° C. for 45-70 minutes, preferably 60 minutes.

In one embodiment of the invention, the filtration in step f of the method is carried out with a rotary drum filter. The filter pore size here is 0.4-0.6 mm, and preferably 0.5 mm.

In one embodiment of the invention, the pasteurization process in step g of the method is carried out at 80-100° C., preferably 90-95° C., for 40-80 seconds and preferably 60 seconds. Then, the product is cooled to 30-70° C. and preferably 40-55° C. for the enzymatic step.

In one embodiment of the invention, the enzymatic process performed in step h in the method is carried out with the pectinase enzyme.

In one embodiment of the invention, the clarification process in step i in the method is carried out with bentonite.

In one embodiment of the invention, the cooling carried out in step n in the method is carried out with a plate cooler.

### DETAILED DESCRIPTION

At least one embodiment of the present invention relates to a method for producing prune juice concentrate comprising the following steps:
a. supplying prunes,
b. subjecting the prunes to the cleaning process,
c. softening the cleaned prunes by adding water,
d. conveying the products obtained in step c to the mash tank,
e. obtaining raw fruit juice by pressing the products conveyed to the press from the mash tank,
f. applying coarse filtration to the juice obtained in step e,
g. carrying out the pasteurization process
h. carrying out the enzymatic process,
i. carrying out the clarification process,
j. carrying out the rotary vacuum filtration,
k. carrying out ultrafiltration,
l. taking the filtered product into clear tanks,
m. carrying out the evaporation,
n. cooling the concentrated product obtained in step m,
o. mixing the cooled product,
p. performing the filling.

In industrial fruit juice production, the purity of the product, the acidity value (pH value), and the cost of the production method are the most important criteria. The fewer chemical products are used during production, the healthier and purer the product obtained, and thus it becomes preferred by the consumer. At least one embodiment of the present invention provides a cost-effective method for the production of high-purity prune juice concentrate.

In one embodiment of the invention, the plums used in the method of the invention are sweet plums. The mentioned plums are selected from a list consisting of President, Stanley, Sugar, Uryani, or a combination of these.

In one embodiment of the method of the present invention, the prunes to be processed first pass the suitability tests. Then, the raw material passes through the selection band and is washed after being purified from foreign materials.

In one embodiment of the invention, the amount of water in step c of the method is twice the amount of the mentioned prune by weight.

In one embodiment of the invention, the softening process in step c of the method is carried out by heating the mentioned prunes at 50-80° C., preferably 60-70° C. for 100-140 minutes and preferably 120 minutes. Thus, the plums are sufficiently softened. Sufficiently softened prunes were sent to the mash tanks by means of pumps.

In one embodiment of the invention, the products conveyed to the mash tank in step d of the method are kept here at 40-70° C., preferably 50-60° C. for 45-70 minutes, preferably 60 minutes. Raw prune juice is obtained by pressing the products conveyed to the press from the mash tank. BUCHER unipektin AG HPX 7507 is used as a press in at least one embodiment of the present invention. After the press, the remaining pulp is thrown out by means of a spiral.

In one embodiment of the invention, the filtration in step f of the method is carried out with a rotary drum filter. Thus, large particles are removed before pasteurization and ultrafiltration. The filter pore size here is 0.4-0.6 mm, and preferably 0.5 mm.

The prune juice coming out of the rotary drum filter is sent to the raw juice tanks.

In one embodiment of the invention, the pasteurization process in step g of the method is carried out at 80-100° C., preferably 90-95° C., for 40-80 seconds, preferably 60 seconds. Then, the product is cooled to 30-70° C. and preferably 40-55° C. for the enzymatic process step.

In one embodiment of the invention, the product sent to the enzyme tanks is subjected to the enzymatic process in the h step of the method. Here, pectinase (Rapidase Pro Color, DSM Food Specialties B.V.) enzyme is added to the medium at a rate of 40-60 ml/ton, preferably 50 ml/ton.

The process performed by adding pectinase enzyme to breakdown the dissolved pectin in prune juice in the first step of clarification in step i of at least one embodiment of the method of the invention is called depectinization. Whether the depectinization is complete is checked with an alcohol test. An alcohol test is performed on the sample taken from the tank. In step i, 0.4-0.6 kg/ton, preferably 0.5 kg/ton bentonite (CLARIT® UF-CLARIANT, Clariant Produkte GmbH) is added to the tank, which reaches the appropriate value as a result of the alcohol test, and the clarification process is completed.

In one embodiment of the invention, the cooling performed in step n in the method is performed with a plate cooler. In this way, a low-cost method of obtaining prune juice concentrate is offered.

### Example—Production of Prune Juice Concentrate

Prunes were supplied under suitable conditions. Prune raw material compatibility tests were carried out. The raw material accepted to the factory is passed through the selection band and washed after being purified from impurities. Twice the weight of water was added to 1200 kg of prunes. The plums taken into the extraction tanks were heated at 60-70° C. for 120 minutes. Therefore, the plums were sufficiently softened. The fruits, which were treated with water and softened well, were sent to the mash tanks by means of pumps. The products sent to the mash tank were pressed after waiting for 60 minutes at 50-60° C. The fruits were pressed by sending them to the Press (BUCHER unipektin AG HPX 7507) from the mash tanks. The remaining pulp was thrown out by means of a spiral. The prune juice coming out of the press was sent to the rotating drum filter and passed through coarse filtration. The filter pore size used here is 0.5 mm. The prune juice coming out of the rotary drum filter was sent to the raw juice tanks. The product was sent to the pasteurizer for pasteurization. Prune juice was pasteurized at 90-95° C. for 60 seconds, and then cooled to 40-55° C. for enzymatic process and sent to the enzyme tanks. The product sent to the enzyme tanks was subjected to the enzymatic process. Here, pectinase (Rapidase Pro Color, DSM Food Specialties B.V.) enzyme was added at a rate of 50 ml/ton. The completion of depectinization was checked with an alcohol test. An alcohol test was performed on the sample taken from the tank. The following steps are performed for the alcohol test:
An acidic alcohol solution is prepared by carefully adding 5 ml of hydrochloric acid (HCL) to 245 ml of ethyl alcohol (EtOH).
The sample taken from the enzyme tank is passed through filter paper,
5 ml of the filtered sample is taken, put into the test tube, and 10 ml of ethyl alcohol-HCl solution is added,
By inverting the test tube 2-3 times, the sample is mixed with the alcohol solution and interpreted.
After the sample is kept for 5-10 minutes, the results are interpreted.
The result is deemed positive if air bubbles and flocculation are not visible.

The clarification process was carried out by adding 0.5 kg/ton bentonite (CLARIT® UF-CLARIANT, Clariant Produkte GmbH) which was prepared before to the tank, which reaches the appropriate value as a result of the alcohol test. The clarified product from the enzyme tanks was pre-filtered with the aid of a perlite-wrapped vacuum filter (Vacuum Rotary Filter TMCI Padovan-Italien Type: Taylo). The product coming out of the rotary vacuum filter was sent to the Ultra filter (Bucher Unipektin AG, UltraStar XL) to be filtered. Turbidity analyzes were performed per hour. The filtered product was taken into clear tanks before being sent to the evaporator. The filtered product in the clear tanks was sent to the evaporator (ING. A. ROSSI, Evaporator 6T 13000 TC). A product with 70 brix was obtained by evaporating water. The concentrated product was brought to 10-20° C. by passing through the plate cooler before going to the concentrated batch tank. The concentrated product was taken into the batch tank to become homogeneous. It was mixed with the help of a mixer and it was ensured to be homogeneous. The product was filled in aseptic bags, labeled in 275 kg conical barrels, and taken to the cold storage at −18° C.

The scope of protection of the invention is specified in the appended claims and cannot be limited to what is described in this detailed description for exemplary purposes. As it is clear that a person skilled in the art can present similar embodiments in the light of what has been described above without departing from the main theme of the invention.

1. A production method for prune juice concentrate, the method comprising:
(a) supplying prunes,
(b) subjecting the prunes to a cleaning process,
(c) softening the prunes by adding water,
(d) conveying the products obtained in step (c) to a mash tank,
(e) obtaining raw fruit juice by pressing the products from the mash tank, wherein the products from the mash tank are conveyed to a press,
(f) applying coarse filtration to the raw fruit juice obtained in step (e),
(g) carrying out a pasteurization process,
(h) carrying out an enzymatic process,
(i) carrying out a clarification process,
(j) carrying out rotary vacuum filtration,
(k) carrying out ultrafiltration,
(l) taking the filtered product into clear tanks,
(m) carrying out evaporation,
(n) cooling the concentrated product obtained in step (m),
(o) mixing the cooled product, and
(p) performing filling.

2. The method according to claim 1, wherein an amount of the water in step (c) is twice by weight of an amount of the prunes.

3. The method according to claim 1, wherein the softening process in step (c) is carried out by heating the plums at 50-80° C. for 100-140 minutes.

4. The method according to claim 1, wherein the products conveyed to the mash tank in step (d) are kept in the mash tank at 40-70° C. for 45-70 minutes.

5. The method according to claim 1, wherein the filtration in step (f) is carried out with a rotary drum filter.

6. The method according to claim 5, wherein a filter pore size of the rotary drum filter is 0.4-0.6 mm.

7. The method according to claim 1, wherein the pasteurization process in step (g) is carried out at 80-100° C. for 40-80 seconds.

8. The method according to claim 1, wherein the product obtained in step (g) is cooled down to 30-70° C. for the enzymatic process.

9. The method according to claim 1, wherein the enzymatic process performed in step (h) is performed with a pectinase enzyme.

10. The method according to claim 1, wherein the clarification process in step (i) is carried out with bentonite.

11. The method according to claim 1, wherein the cooling performed in step (n) is carried out with a plate cooler.
